(19) United States
(12) Patent Application Publication
(10) Pub. No.: US 2023/0078072 A1
(43) Pub. Date: Mar. 16, 2023

(54) System For Treating Neurological Drive Dysfunction

(71) Applicant: KiHealthConcepts LLC, Hernando, FL (US)

(72) Inventor: Mark David Warren, Hernando, FL (US)

(73) Assignee: KiHealthConcepts LLC, Hernando, FL (US)

(21) Appl. No.: 17/412,467

(22) Filed: Aug. 26, 2021

(51) Int. Cl. G16H 20/70; G06N 5/02

(52) U.S. Cl. CPC G16H 20/70; G06N 5/02

(57) ABSTRACT

An example neurological drive dysfunction (NDD) treatment system may include one or more of a processor of a server node connected to at least one user mobile node over a network; a memory on which are stored machine readable instructions that when executed by the processor, cause the processor to: receive from the user mobile node neurological drive dysfunction (NDD) data; provide the received NDD data to an AI module executed on the server node; send an NDD category selected by the AI module to the user mobile node; receive new NDD findings data from the user mobile node; provide the new NDD findings data to the AI module; and send a new NDD category recommendation generated by the AI module based on the new NDD findings.

### TECHNICAL FIELD

This application generally relates to correcting neurological drive dysfunction, and more particularly, to a non-invasive method and system for correcting neurological drive dysfunction using techniques grouped by the category of neurological drive dysfunction.

### BACKGROUND

Neurological drive dysfunction is common among patients of all ages. Neurological dysfunction refers to a disorder of the Central Nervous System (CNS) which affects the efficiency and effectiveness of reflexive processes of the CNS. All people have a degree of neurological dysfunction, while in the majority of people the dysfunctions are minimal.

Optimally the nervous system homogenizes the level of neurological drive of a motor neuron pool to evenly spread the work load through the system, resulting in a decreased chance of injury. However, sections of the motor neuron pool become unhomogenized, resulting in some motor neurons continuing to supply a higher neurological drive than the rest. This situation is referred to as a neurological drive dysfunction. The neurological drive dysfunction creates observable signs and an increased chance of injury to both the nervous system and soft tissue structures it supplies.

Available treatments of the neurological drive dysfunction may range from medications such as the neuroleptics (e.g., haloperidol, chlorpromazine, baclofen, diazepam, methocarbamol and tizanidine) used to treat organic disorders of the brain such as schizophrenia, to comparatively simple analgesics, such as ibuprofen, acetaminophen and opiates to treat the painful effects of many neurological ailments. Most of the conventional treatments are either ineffective or produce only short term results.

Accordingly, what is needed is a non-invasive effective method and system for correcting neurological drive dysfunction using techniques grouped by the category of neurological drive dysfunction being targeted.

### SUMMARY

An example embodiment provides a system for treatment of neurological drive dysfunction that includes one or more of a processor of a server node connected to at least one user mobile node over a network; a memory on which are stored machine readable instructions that when executed by the processor, cause the processor to: receive from the user mobile node neurological drive dysfunction (NDD) data; provide the received NDD data to an AI module executed on the server node; send an NDD category selected by the AI module to the user mobile node; receive new NDD findings data from the user mobile node; provide the new NDD findings data to the AI module; and send a new NDD category recommendation generated by the AI module based on the new NDD findings.

Another example embodiment provides a method for treatment of neurological drive dysfunction that includes one or more of receiving from the user mobile node neurological drive dysfunction (NDD) data; providing the received NDD data to an AI module executed on the server node; sending an NDD category selected by the AI module to the user mobile node; receiving new NDD findings data from the user mobile node; providing the new NDD findings data to the AI module; and sending a new NDD category recommendation generated by the AI module based on the new NDD findings to the user mobile node.

### BRIEF DESCRIPTION OF THE DRAWINGS

FIG. 1 illustrates a conceptual overview of a system flow including an AI module, according to example embodiments;

FIG. 2 illustrates flowchart of a method for correcting neurological drive dysfunction, according to example embodiments;

FIGS. 3A and 3B illustrate further flowcharts of a method for a method for correcting neurological drive dysfunction, according to example embodiments;

FIGS. 4A and 4B illustrate further flowcharts of a method for correcting neurological drive dysfunction, according to example embodiments;

FIGS. 5A and 5B illustrate further flowcharts of a method for correcting neurological drive dysfunction, according to example embodiments.

FIG. 6A illustrates a network architecture of an NDD treatment that may be used by the exemplary embodiments.

FIG. 6B illustrates a further NDD treatment system that uses an input from an AI system based on data retrieved from a blockchain 621, according to example embodiments.

FIG. 7 illustrates an example of a blockchain which stores machine learning (AI) data, according to example embodiments.

FIG. 8 illustrates network architecture of a NDD treatment system and detailed functionality of a server node that may be used by the example embodiments.

FIG. 9 illustrates a flowchart of a method executed by the server node, according to example embodiments.

FIG. 10 illustrates an example computer/server node that supports one or more of the example embodiments.

### DETAILED DESCRIPTION

It will be readily understood that the instant components, as generally described and illustrated in the figures herein, may be arranged and designed in a wide variety of different configurations. Thus, the following detailed description of the embodiments of at least one of a method, apparatus, non-transitory computer readable medium and system, as represented in the attached figures, is not intended to limit the scope of the application as claimed but is merely representative of selected embodiments.

The instant features, structures, or characteristics as described throughout this specification may be combined or removed in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment. Thus, appearances of the phrases “example embodiments”, “in some embodiments”, “in other embodiments”, or other similar language, throughout this specification do not necessarily all refer to the same group of embodiments, and the described features, structures, or characteristics may be combined or removed in any suitable manner in one or more embodiments. Further, in the diagrams, any connection between elements can permit one-way and/or two-way communication even if the depicted connection is a one-way or two-way arrow. Also, any device depicted in the drawings can be a different device. For example, if a mobile device is shown sending information, a wired device could also be used to send the information.

In addition, while the term “message” may have been used in the description of embodiments, the application may be applied to many types of networks and data. Furthermore, while certain types of connections, messages, and signaling may be depicted in exemplary embodiments, the application is not limited to a certain type of connection, message, and signaling.

Example embodiments provide methods, systems, components, non-transitory computer readable media, devices, and/or networks, which provide for a non-invasive system and method for correcting neurological drive dysfunction based on techniques that are grouped by the category of the neurological drive dysfunction they target and the type of neurological summation they are designed to achieve.

A system and method, according to the exemplary embodiments, overcome the aforementioned problems and disadvantages of conventional treatments of the neurological drive dysfunction by providing a system of techniques that directly and indirectly stimulate mechanoreceptors that feed into four of the nervous system inhibition systems and activate one of two types of neurological summation.

In doing so the system lowers the neurological drive of the targeted motor neurons, homogenizing the neurological drive and workload over the entire motor neuron pool and decreasing the chances of injury. An advantage of the present approach is that the system of linked techniques and feedback loops acts as a guide to the practitioner, leading them to the most effective techniques for the specific neurological drive dysfunction category. Another advantage of the exemplary embodiments is that the system of linked techniques and feedback loops guides the practitioner to the most efficient technique first, saving them both time and energy.

A further advantage of the exemplary embodiments is that, by creating a system that quickly guides the practitioner to the most effective and efficient techniques, means that the patient will always receive the minimum effective dose, increasing the speed to completion within the system and decreasing the chances of its overuse. Another advantage of the exemplary system that is each technique's specific five step application strategy, giving the practitioner a repeatable algorithm to follow for consistent results. Yet another advantage is the versatility of the system, which gives the practitioner a choice of tools they may use. The size, shape, design and material of the device that the practitioner uses should be considered separate to, and not a part of, the present application. It is only important to the system that the device is able to successfully perform each step of the technique's application strategy. Whilst neurological drive dysfunction can result in injury to nerves and myofascial units, the treatment of the diseased or injured nerves, muscles, tendons, fascia and the adhesion or scar tissue thereof are considered separate to, and not a part of, the present application as the system itself is purely focused on stimulating a change of behavior within a healthy nervous system.

In one embodiment, the neurological drive dysfunction (NDD) category may be acquired from an artificial intelligence (AI) system based on parameters of the patient observed by a practitioner. In one embodiment, the treatment recommendations may be predicted by an AI system model that uses data retrieved from a decentralized storage such as a blockchain.

The decentralized storage may include an append-only immutable data structure resembling a distributed ledger capable of maintaining and records between mutually untrusted parties. The untrusted parties are referred to herein as peers or peer nodes. Each peer maintains a copy of the NDD records and patient's parameters and no single peer can modify the records without a consensus being reached among the distributed peers. For example, the peers may execute a consensus protocol to validate blockchain storage transactions, group the storage transactions into blocks, and build a hash chain over the blocks. This process forms the ledger by ordering the storage transactions, as is necessary, for consistency. In various embodiments, a permissioned and/or a permissionless blockchain can be used. In a public or permissionless blockchain, anyone can participate without a specific identity. Public blockchains can involve native cryptocurrency and use consensus based on various protocols such as Proof of Work (PoW). On the other hand, a permissioned blockchain provides secure interactions among a group of entities which share a common goal such as donating and collecting funds for a common charitable cause, but which do not fully trust one another.

Accordingly, the example embodiments provide for a specific solution to a problem in the field of medical treatments of the neurological drive dysfunction. According to the exemplary embodiments, the programmable system coupled to an AI module may be used for effective treatment of the neurological drive dysfunction (NDD).

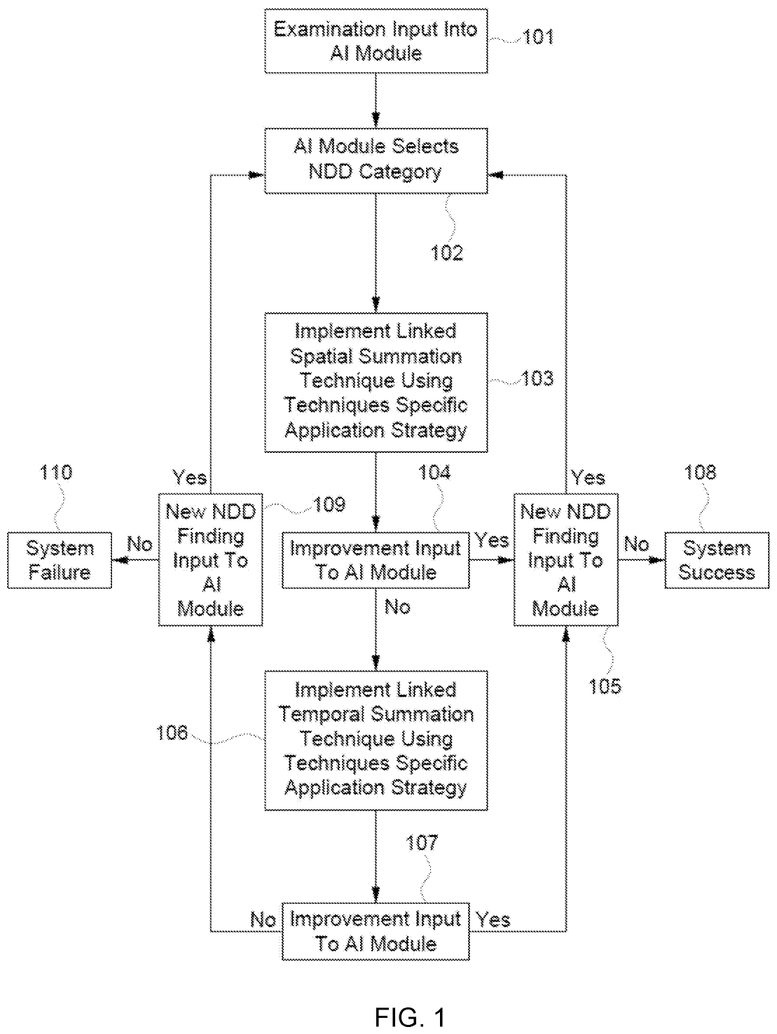

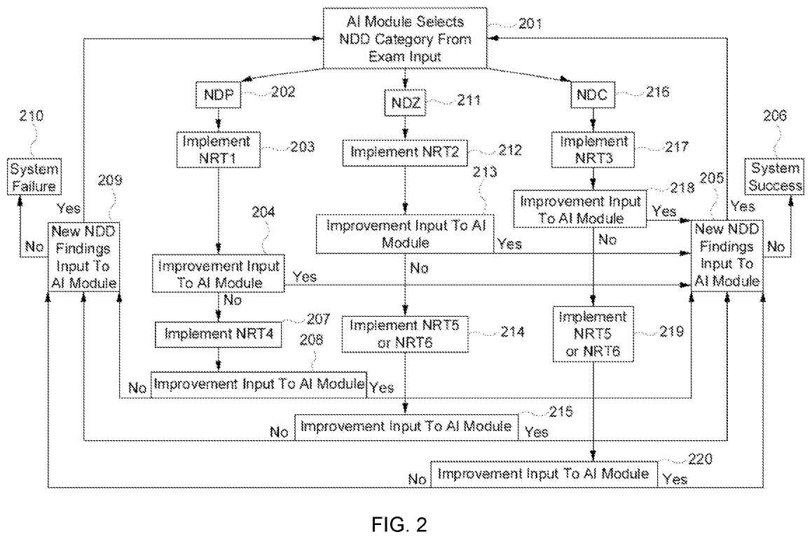

FIG. 1 illustrates a conceptual overview of the workflow of the system, according to example embodiments. FIG. 1 depicts a general overview of the system workflow created to give a broader and simpler flow through the process before added layers of detail are reviewed in FIGS. 2-5.

At block 101, a practitioner inputs the examination data into the system and the AI module may process the examination data and may recommend the category of Neurological Drive Dysfunction (NDD) at block 102. At block 103, out of the six Neurological Response Techniques (NRT1-6), three (NRT1-3) were selected by the AI model to take advantage of Spatial Summation. The AI module may initially prioritize these techniques to be performed first as they are more time and energy efficient than the three techniques (NRT4-6) that were created to take advantage of Temporal Summation. The AI system may inform the practitioner which of these techniques to use and how to use them with text, picture and/or video instruction. In one embodiment, the AI system may provide the instructions to the practitioner's smartphone or tablet.

After completion of the technique indicated at block 103, the AI module may prompt the practitioner to input into the system if there has been sufficient improvement to be entered at block 104. If the answer is yes, the system may then prompt the practitioner to input if there are any new NDD findings 105. If there is the AI module will analyze the data, select the new NDD category at block 102 and instruct the practitioner to proceed through the system again as described at block 103 by guiding him through the application of its linked Spatial Summation technique. However, if the practitioner inputs into the system at block 105 that there are no new NDD findings, the AI module will consider this a successful result at block 108 and may instruct the practitioner to terminate the procedure.

Alternatively, if the practitioner inputs into the system that there is insufficient improvement at block 104, the AI module will inform the practitioner which of the Temporal Summation techniques at block 106 to use and how to use it via text, pictures and/or video instruction provided to his smartphone or tablet. After completion of the techniques at block 106, the AI module will prompt the practitioner to input into the system if there has been sufficient improvement at block 107. If the answer at block 107 is yes, the system will prompt the practitioner to input if there are any new NDD findings at block 105. If there are new NDD findings, the AI module may analyze the data, select the new NDD category at block 102 and may instruct the practitioner to proceed through the system again as described at block 103 by guiding the practitioner through the application of its linked Spatial Summation technique.

However, if the practitioner inputs into the system at block 105 that there are no new NDD findings, the AI module will consider this a successful result at block 108 and may instruct the practitioner to terminate the process. Alternatively, if the answer at block 107 is no, the system may also prompt the practitioner to input if there are any new NDD findings at block 109. If there are new NDD findings, the AI module may analyze the data, select the new NDD category at block 102 and may instruct the practitioner to proceed through the system again as described at block 103 by guiding the practitioner through the application of its linked Spatial Summation technique. However, if the practitioner inputs into the system at block 109 that there are no new NDD findings, the AI module may consider this a failed result at block 110 and may instruct the practitioner to terminate the process.

FIG. 2 illustrates flowchart of a method for correcting neurological drive dysfunction, according to example embodiments.

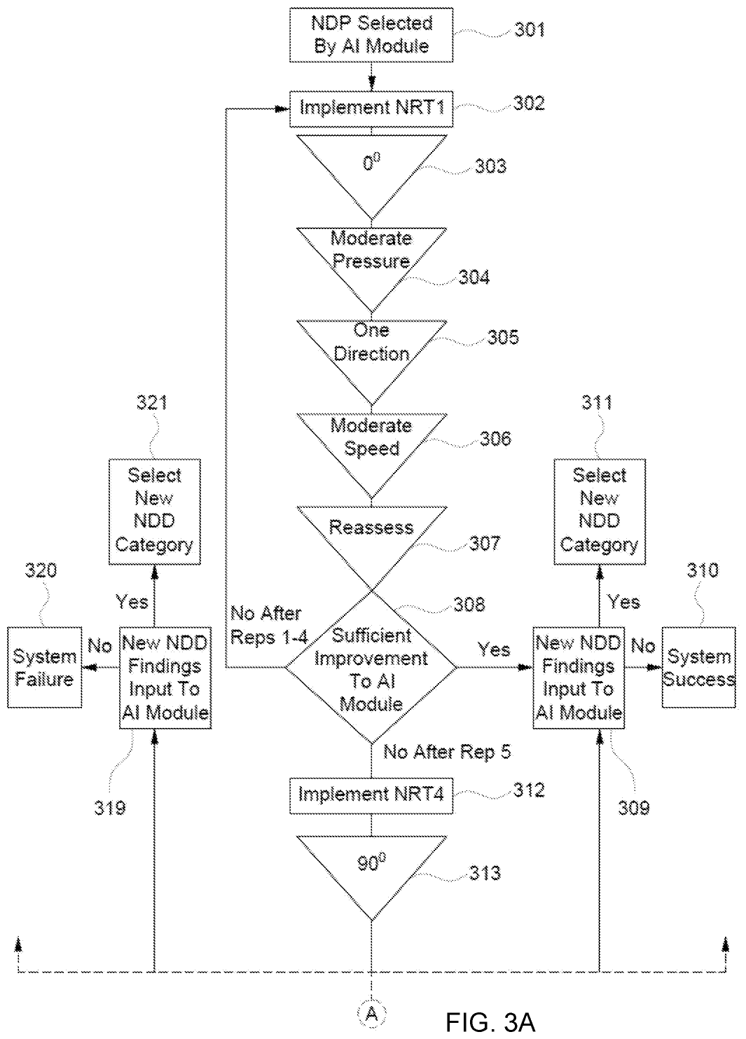

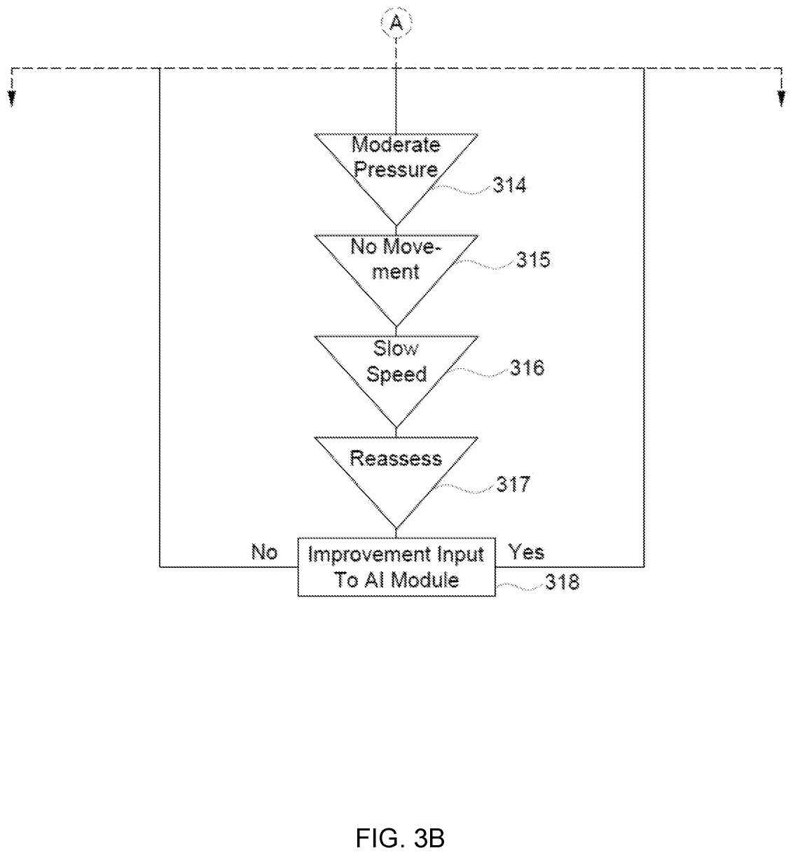

Once NDD has been observed, the practitioner enters the observation-related data into the system and the AI module selects, block 201, one appropriate NDD category from three available based on the observation-related data. According to one embodiment, if a Neurological Drive Point (NDP) at block 202 is recommended by the AI module, the practitioner implements Neurological Response Technique 1 (NRT1) at block 203 using its specific five step application strategy, the order of which is adhered to exactly as shown in FIG. 3 to be considered complete.

If sufficient improvement is made at block 204 and new NDD findings were present on reassessment at block 205, then the practitioner provides the new NDD findings to the AI module that selects the new NDD category at block 201 and the practitioner continues through the system. If sufficient improvement is made at block 205, and there are no new NDD findings at block 205, this is considered a successful result at block 206 and the treatment process is terminated. If insufficient improvement is made at block 204, the practitioner implements Neurological Response Technique 4 (NRT4) at block 207 using its specific five step application strategy, the order of which must be adhered to exactly as shown in FIG. 3 to be considered complete. If sufficient improvement is made at block 208, and new NDD findings were present on reassessment at block 205, then the practitioner provides the new NDD findings to the AI module that selects the new NDD category at block 201 and the practitioner continues through the treatment system. If sufficient improvement is made at block 208, and there are no new NDD findings at block 205, this is considered a success at block 206 and the treatment process is terminated.

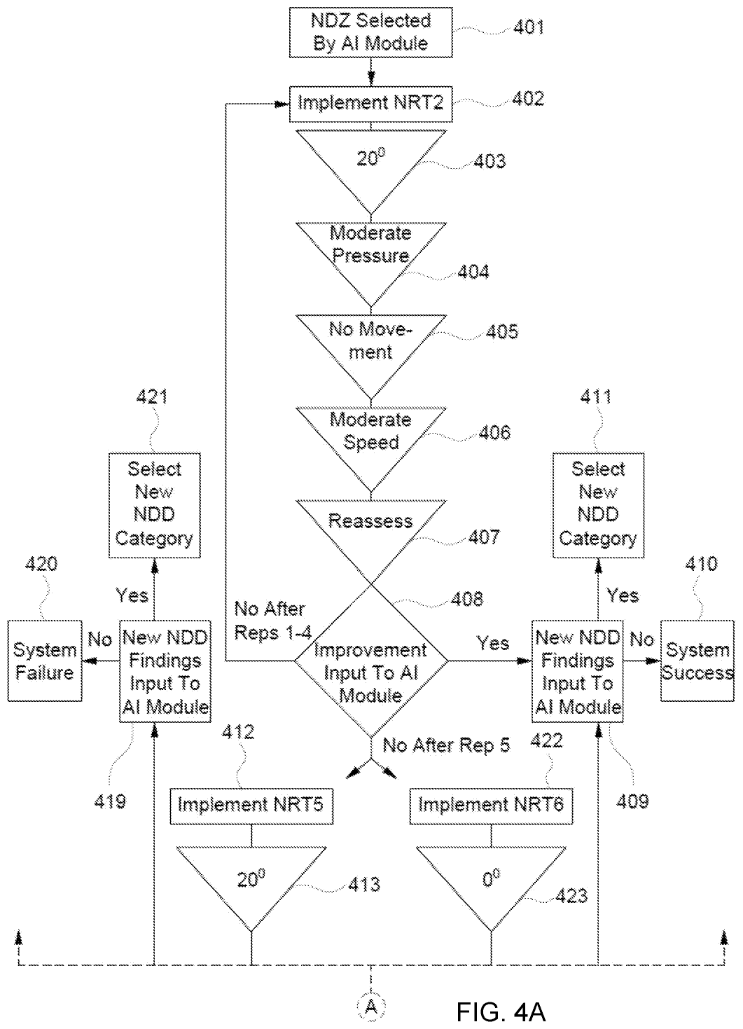

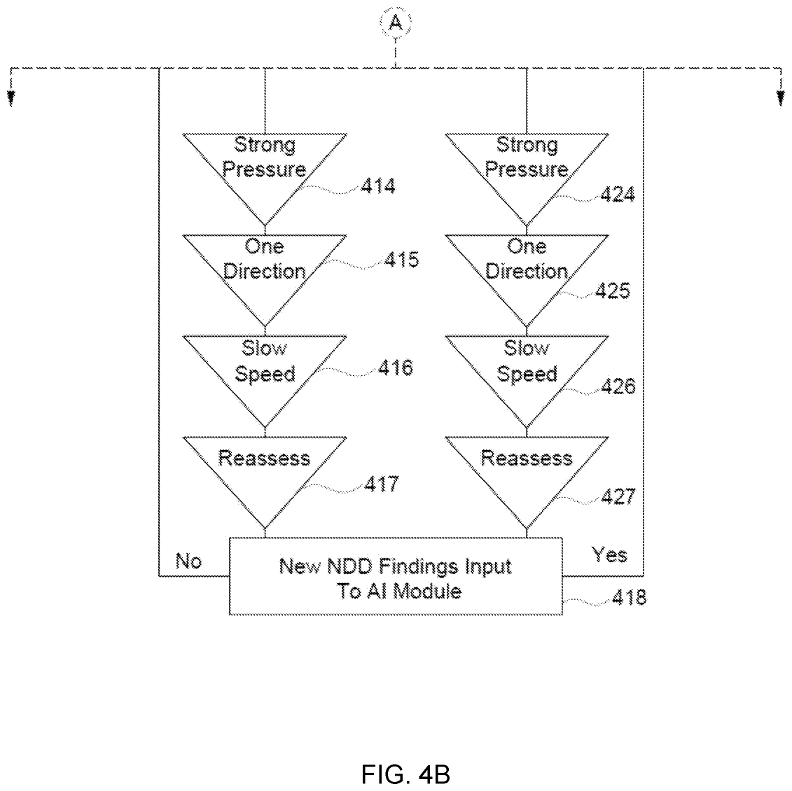

If insufficient improvement is made at block 208, and new NDD findings were present on reassessment at block 209, the practitioner provide the new NDD findings to the AI module that selects the new NDD category at block 201 and the practitioner continues through the treatment system. If insufficient improvement is made at block 208, and there are no new NDD findings at block 209, this is consider a failure at block 210 and the treatment process is terminated. According to the exemplary embodiments, if a Neurological Drive Zone (NDZ) at block 211 is selected by the AI module, the practitioner implements Neurological Response Technique 2 (NRT2) at block 212 using its specific five step application strategy, the order of which must be adhered to exactly as shown in FIG. 4 to be considered complete. If sufficient improvement is made at block 213, and new NDD findings were present on reassessment at block 205, the practitioner provides the new NDD findings to the AI module that selects the new NDD category at block 201 and the practitioner continues through the treatment system. If sufficient improvement is made at block 213, and there are no new NDD findings at block 205, this is considered successful at block 206 and the treatment process is terminated. If insufficient improvement is made at block 213, the practitioner implements Neurological Response Technique 5 (NRT5) or Neurological Response Technique 6 (NRT6) at block 214 depending on the body region being treated. Each of these techniques have their respective five step application strategy, the order of which must be adhered to exactly as shown in FIG. 4 to be considered complete. If sufficient improvement is made at block 215, and new NDD findings were present on reassessment at block 205, the practitioner provided the NDD findings to the AI module that selects the new NDD category at block 201 and the practitioner continues through the treatment system. If sufficient improvement is made at block 215 and there are no new NDD findings at block 205, this is considered successful at block 206 and the treatment system is terminated.

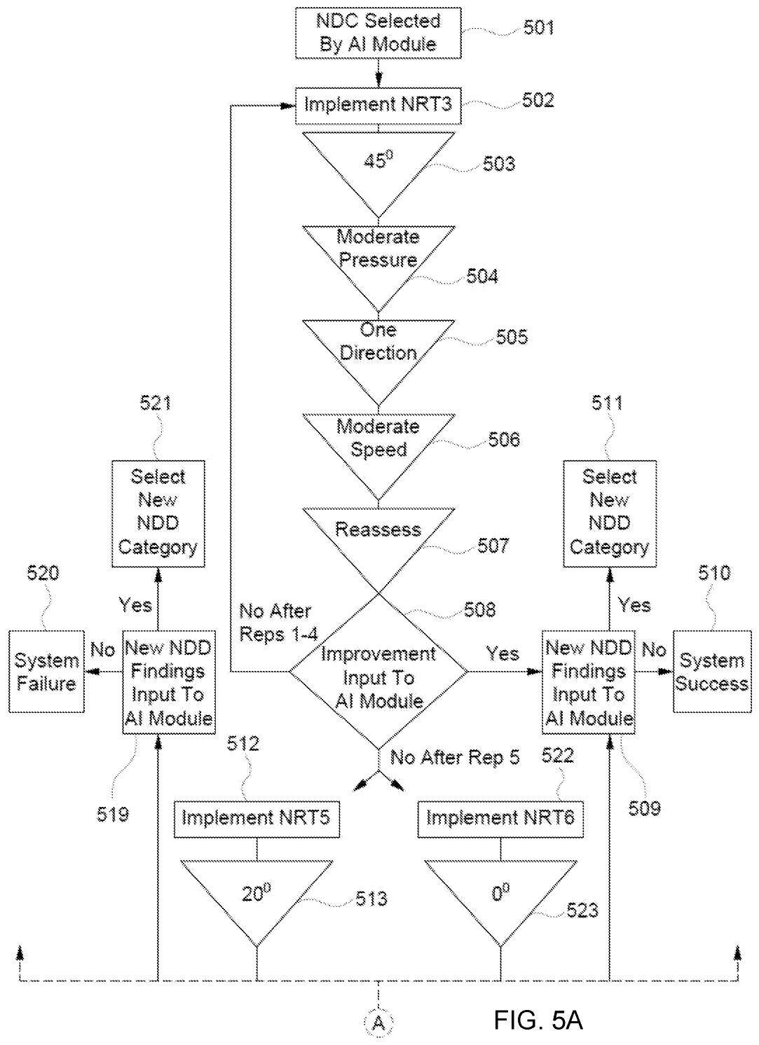

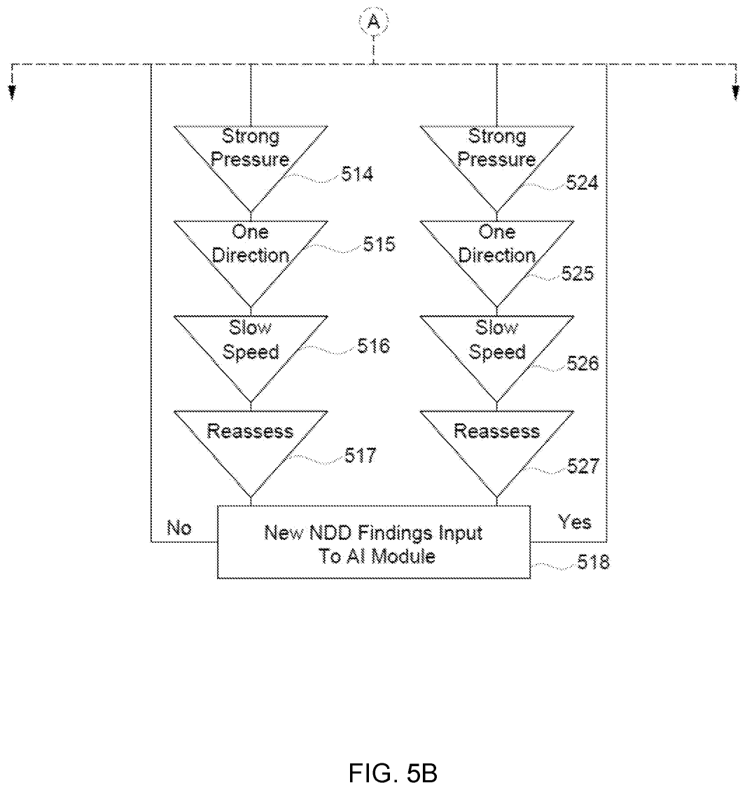

If insufficient improvement is made at block 215, and new NDD findings were present on reassessment at block 209, the practitioner provides the new NDD findings to the AI module that selects the new NDD category at block 201 and the practitioner continues through the treatment system. If insufficient improvement is made at block 215, and there are no new NDD findings at block 209, this is consider a failure at block 210 and the treatment process is terminated. According to the exemplary embodiments, if a Neurological Drive Chain (NDC) at block 216 is recommended by the AI module, the practitioner implements Neurological Response Technique 3 (NRT3) at block 217 using its specific five step application strategy, the order of which is adhered to exactly as shown in FIG. 5 to be considered complete.

If sufficient improvement is made at block 218, and new NDD findings were present on reassessment at block 205, the practitioner provides the new NDD findings to the AI module that selects the new NDD category at block 201 and the practitioner continues through the treatment system. If sufficient improvement is made at block 218, and there are no new NDD findings at block 205, this is considered successful at block 206 and the treatment process is terminated. If insufficient improvement is made at block 218, the practitioner implements Neurological Response Technique 5 (NRT5) or Neurological Response Technique 6 (NRT6) at block 219 depending on the body region being treated. Each of these techniques has their respective five step application strategy, the order of which is adhered to exactly as shown in FIG. 5 to be considered complete. If sufficient improvement is made at block 220, and new NDD findings were present on reassessment at block 205, the practitioner enters the new NDD findings into the AI module that selects the new NDD category at block 201 and continues through the system. If sufficient improvement is made at block 220, and there are no new NDD findings at block 205, this is considered successful at block 206 and the treatment process is terminated. If insufficient improvement is made at block 220, and new NDD findings were present on reassessment at block 209, the practitioner provides the new NDD findings to the AI module that selects the new NDD category at block 201 and the practitioner continues through the treatment system. If insufficient improvement is made at block 220, and there are no new NDD findings at block 209, this is consider a failure at block 210 and the treatment process is terminated.

According to the exemplary embodiments, if Neurological Drive Point (NDP) is selected at block 301 as the appropriate NDD category, the practitioner implements its linked spatial summation technique, Neurological Response Technique 1 (NRT1) at block 302, using its specific five step application strategy. Those five steps for NRT1 are applied in the exact sequential order they appear in FIG. 3 for the technique to be considered complete. At block 303 (in step 1), the selected device is placed at a zero degree “flat” angle over the target mechanoreceptors. At bock 304 (in step 2), moderate pressure of 10-20 Newtons is applied over the targeted mechanoreceptors. At block 305 (step 3), movement in one direction of the treatment device at 10 mm per second over the target mechanoreceptors is performed. At block 306 (step 4), treatment device applied at a moderate speed taking 2-10 seconds to complete the technique.

At block 307 (step 5), reassessment of the NDD to assess for changes or note new findings is performed. If sufficient improvement is made at block 308, and new NDD findings were present on reassessment at block 309, the practitioner submits the new NDD findings to the AI module and receives new NDD category at block 311 and implements the linked Neurological Response Techniques, as shown in FIG. 2. If sufficient improvement is made at block 308, and there are no new NDD findings at block 309, this is considered successful at block 310 and the treatment process is terminated. If insufficient improvement is made at block 308 after repetitions 1-4 of implementing NRT1, the practitioner applies a further repetition of the technique, taking them back through the treatment system from blocks 302-308. However, if insufficient improvement still exists after the fifth repetition of applying NRT1, the practitioner moves on to implementing the NDPs linked temporal summation technique, Neurological Response Technique 4 (NRT4) at block 312. The five step application strategy for NRT4 is implemented in the exact sequence depicted in FIG. 3 for the technique to be considered complete. At block 313 (step 1), the selected device is placed at a ninety degrees contact over target mechanoreceptors. At block 314 (step 2), moderate pressure of 10-20 Newtons is applied over the targeted mechanoreceptors. At block 315 (step 3), no movement of the device occurs over target mechanoreceptors. At block 316, (step 4), the technique is applied at a slow speed taking 30-180 seconds to complete. At block 317 (step 5), reassessment of the NDD to assess for changes or note new findings is performed. If sufficient improvement is made at block 318, and new NDD findings were present on reassessment at block 309, the practitioner submits the new NDD findings to the AI module that selects the new NDD category 311. The practitioner then implements the linked Neurological Response Techniques, as shown in FIG. 2. If sufficient improvement is made at block 318, and there are no new NDD findings at block 309, this is considered successful at block 310 and the treatment method is terminated. If insufficient improvement is made at block 318, and new NDD findings were present on reassessment at block 319, the practitioner provides the NDD findings to the AI module and receives the new NDD category selected by the AI module at block 321. The practitioner then implements the linked Neurological Response Techniques, as shown in FIG. 2. If insufficient improvement is made at block 318, and there are no new NDD findings at block 319, this is consider a failure at block 320 and the treatment process is terminated.

According to the exemplary embodiments, if Neurological Drive Zone (NDZ) is selected at block 401 as the appropriate NDD category, the practitioner implements its linked spatial summation technique, Neurological Response Technique 2 (NRT2) at block 402, using its specific five step application strategy. Those five steps for NRT2 are applied in the exact sequential order they appear in FIG. 4 for the technique to be considered complete. At block 403 (step 1), the selected device is placed at a twenty degree angle over the target mechanoreceptors. At block 404 (step 2), moderate pressure of 10-20 Newtons is applied over the targeted mechanoreceptors. At block 405 (step 3), no movement of the device occurs over the target mechanoreceptors. At block 406 (step 4), the devise is applied at a moderate speed taking 2-10 seconds to complete the technique. At block 407 (step 5), reassessment of the NDD to assess for changes or note new findings may be performed.

If sufficient improvement is made at block 408, and new NDD findings were present on reassessment at block 409, the practitioner selects the new NDD category at block 411 and implements the linked Neurological Response Techniques, as shown in FIG. 2. If sufficient improvement is made at block 408, and there are no new NDD findings at block 409, this is considered successful at block 410 and the system is terminated. If insufficient improvement is made at block 408 after repetitions 1-4 of implementing NRT2, the practitioner applies a further repetition of the technique, taking them back through the treatment system from 402-408. However, if insufficient improvement still exists after the fifth repetition of applying NRT2, the practitioner moves on to implement the NDZs linked temporal summation technique of Neurological Response Technique 5 (NRT5) at block 412 or Neurological Response Technique 6 (NRT6) at block 422, depending on which technique is better suited to the body region being treated.

The five step application strategy for NRT5 is implemented in the exact sequence shown in FIG. 4 for the technique to be considered complete. At block 413 (step 1), the selected device is placed at a twenty degree angle to the target mechanoreceptors. At block 414 (step 2), strong pressure of greater than 20 Newtons is applied over the target mechanoreceptors. At block 415 (step 3), movement in one direction of the device at 2 mm per second over target mechanoreceptors is performed. At block 416, (step 4), the technique is applied at a slow speed taking 30-180 seconds to complete. At block 417 (step 5), reassessment of the NDD to assess for changes or note new findings is performed. The five step application strategy for NRT6 is implemented in the exact sequence they appear in FIG. 4 for the technique to be considered complete.

At block 423 (step 1), the selected device is placed at a zero degree angle, “flat” contact, over target mechanoreceptors. At block 424 (step 2), strong pressure of greater than 20 Newtons is applied over the target mechanoreceptors. At block 425 (step 3), initial movement in one direction of the device is followed by a hold over target mechanoreceptors. At block 426 (step), the technique is applied at a slow speed taking 30-180 s to complete. At blocks 427 (step 5), reassessment of the NDD to assess for changes or note new findings is performed. If sufficient improvement is made at 418, and new NDD findings were present on reassessment at block 409, the practitioner submits the new NDD findings to the AI module and received selected new NDD category at block 411. The practitioner then implements the linked Neurological Response Techniques, as shown in FIG. 2. If sufficient improvement is made at block 418, and there are no new NDD findings at block 409, this is considered successful at block 410 and the treatment method is terminated. If insufficient improvement is made at block 418, and new NDD findings were present on reassessment at block 419, the practitioner submits the new NDD findings to the AI module that selects the new NDD category at block 421 and implements the linked Neurological Response Techniques, as shown in FIG. 2. If insufficient improvement is made at block 418, and there are no new NDD findings at block 419, this is considered a failure 420 and the treatment method is terminated.

According to the exemplary embodiments, if Neurological Drive Chain (NDC) is selected at block 501 as the appropriate Neurological Drive Dysfunction (NDD) category, the practitioner implements its linked spatial summation technique, Neurological Response Technique 3 (NRT3) at block 502, using its specific five step application strategy. Those five steps for NRT3 are applied in the exact sequential order depicted in FIG. 5 for the technique to be considered complete. At block 503 (step 1), the selected device is placed at a forty five degree angle over the target mechanoreceptors.

At block 504 (step 2), moderate pressure of 10-20 Newtons is applied over the targeted mechanoreceptors. At block 505 (step 3), movement in one direction of the device at 10 mm per second over the target mechanoreceptors is performed. At block 506 (step 4), the device is applied at a moderate speed taking 2-10 seconds to complete the technique. At block 507 (step 5), reassessment of the NDD to assess for changes or note new findings is performed. If sufficient improvement is made at block 508, and new NDD findings were present on reassessment at block 509, the practitioner provides the new NDD findings to the AI module to selects the new NDD category at block 511. The practitioner then implements the linked Neurological Response Techniques, as shown in FIG. 2. If sufficient improvement is made at block 508, and there are no new NDD findings at block 509, this is considered successful at block 510 and the treatment process is terminated. If insufficient improvement is made at block 508 after repetitions 1-4 of implementing NRT3, the practitioner applies a further repetition of the technique, taking them back through the treatment system from 502-508.

However, if insufficient improvement still exists after the fifth repetition of applying NRT3, the practitioner moves on to implement the NDCs linked temporal summation technique of Neurological Response Technique 5 (NRT5) at block 512 or Neurological Response Technique 6 (NRT6) at block 522, depending on which technique is better suited to the body region being treated. The five step application strategy for NRT5 is implemented in the exact sequence they appear in FIG. 5 for the technique to be considered complete. At block 513 (step 1), the selected device is placed at a twenty degree angle to the target mechanoreceptors. At block 514 (step 2), strong pressure of greater than 20 Newtons is applied over the target mechanoreceptors. At block 515 (step 3), movement in one direction of the device at 2 mm per second over target mechanoreceptors is performed. At block 516 (step 4), the technique is applied at a slow speed taking 30-180 seconds to complete.

At block 517 (step 5), reassessment of the NDD to assess for changes or note new findings is performed. The five step application strategy for NRT6 is implemented in the exact sequence they appear in FIG. 5 for the technique to be considered complete. At block 523 (step 1), the selected device is placed at a zero degree angle, “flat” contact, over target mechanoreceptors. At block 524 (step 2), strong pressure of greater than 20 Newtons is applied over the target mechanoreceptors. At block 525 (step 3), initial movement in one direction of the device is followed by a hold over target mechanoreceptors. At block 526 (step 4), the technique is applied at a slow speed taking 30-180 seconds to complete. At block 527 (step 5), reassessment of the NDD to assess for changes or note new findings is implemented. If sufficient improvement is made at block 518 and new NDD findings were present on reassessment at block 509, the practitioner provides the new NDD findings to the AI module that returns the new NDD category at block 511. The practitioner then implements the linked Neurological Response Techniques, as shown in FIG. 2. If sufficient improvement is made at block 518, and there are no new NDD findings at block 509, this is considered successful at block 510 and the treatment process is terminated.

If insufficient improvement is made at block 518, and new NDD findings were present on reassessment at block 519, the practitioner provides the new NDD findings to the AI module that selects the new NDD category at block 521. The practitioner then implements the linked Neurological Response Techniques, as shown in FIG. 2. If insufficient improvement is made at block 518, and there are no new NDD findings at block 519, this is consider a failure at block 520 and the treatment is terminated.

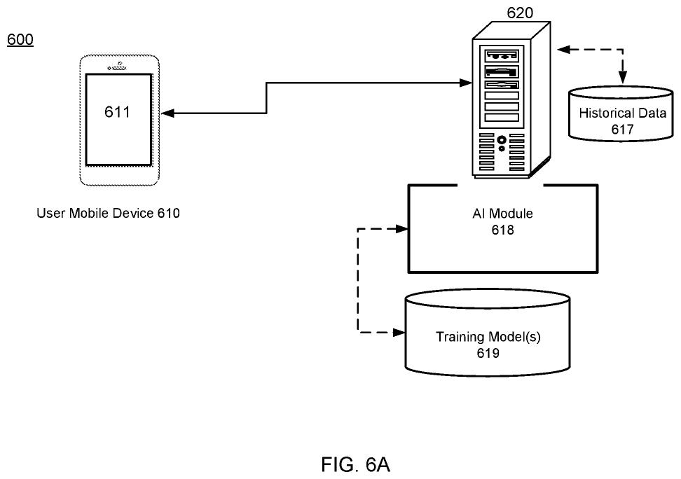

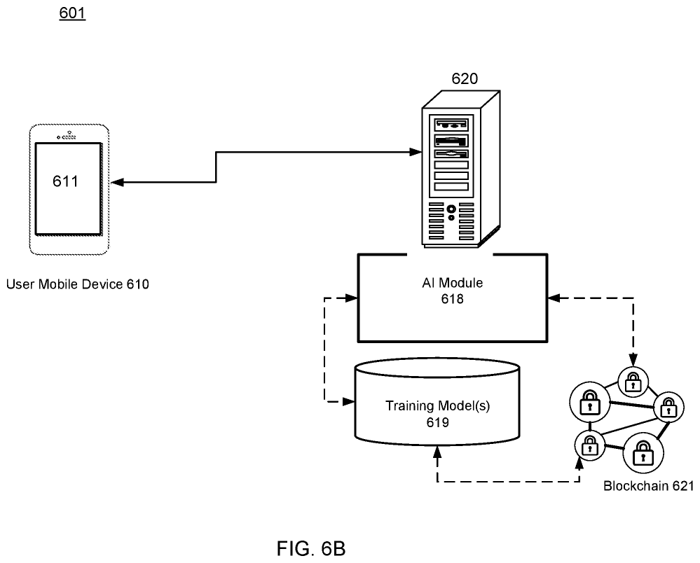

FIG. 6A illustrates a network that may be used by the exemplary embodiments.

As discussed above, the example treatment system 601 may use inputs (e.g., NDD categories) provided by an AI module 618 residing on a server node 620. In one embodiment, the AI system may reside on a cloud. As discussed above, a practitioner may use his or her mobile device 610 (e.g., a smartphone or tablet running a proprietary NDD treatment application 611) to provide NDD finding data to the server node 620 and to receive treatment recommendations. The server node 620 may provide this data to the AI module 618 which may produce NDD treatment recommendations. The treatment recommendations may be acquired from the AI module 618 based on current parameters of a patient (i.e., current NDD findings). In one embodiment, the optimal NDD category may be predicted by the AI training model 119 that uses data retrieved, for example, from a neural network (not shown) or from another source such as a database or blockchain discussed in more details hereafter.

The server node 620 may be a computing device or a server computer, or the like, and may include a processor, which may be a semiconductor-based microprocessor, a central processing unit (CPU), an application specific integrated circuit (ASIC), a field-programmable gate array (FPGA), and/or another hardware device. Although a single processor is intended to be used, it should be understood that the server node 620 may include multiple processors, multiple cores, or the like, without departing from the scope of the server node 620.

The server node 620 may also include a non-transitory computer readable medium that may have stored thereon machine-readable instructions executable by the processor to generate training model(s) 619. Examples of the non-transitory computer readable medium may include an electronic, magnetic, optical, or other physical storage device that contains or stores executable instructions. For example, the non-transitory computer readable medium may be a Random Access Memory (RAM), an Electrically Erasable Programmable Read-Only Memory (EEPROM), a hard disk, an optical disc, or other type of storage device.

FIG. 6B illustrates a further NDD treatment system that uses an input from an AI system based on data retrieved from a blockchain 621, according to example embodiments. As discussed above with reference to FIG. 6A, example treatment system 601 may use inputs (e.g., NDD categories) provided by an AI module 618 residing on a server node 620. In one embodiment, the AI system may reside on a cloud. As discussed above, a practitioner may use his or her mobile device 610 (e.g., a smartphone or tablet running a proprietary NDD treatment application 611) to provide NDD finding data to the server node 620. The server node 620 may provide this data to the AI module 618 which may produce NDD treatment recommendations. The treatment recommendations may be acquired from the AI module 618 based on current parameters of a patient (i.e., current NDD findings). In one embodiment, the optimal NDD category may be predicted by the AI training model 119 that uses data retrieved, for example, from the blockchain 621 configured to store historical data such as NDD findings and previously made treatment recommendations (e.g., NDD categories).

Thus, the NDD treatment system 600/601, advantageously, operates based on the treatment recommendation data using optimal input parameters predicted by the AI module 618, which provides for efficient NDD treatment of patients.

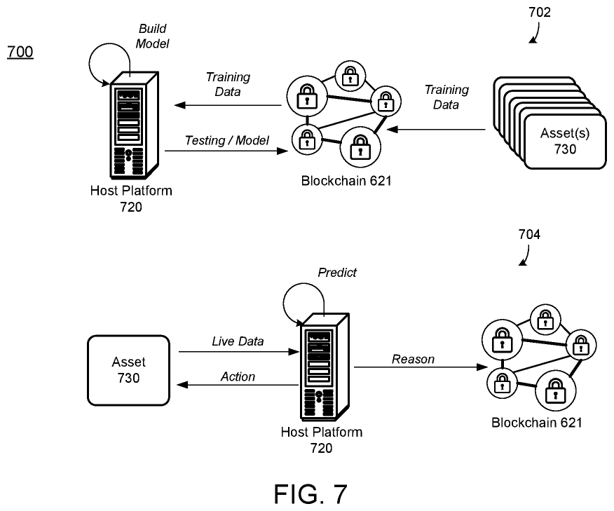

FIG. 7 illustrates an example 700 of a blockchain 621 which stores machine learning (AI) data. Machine learning relies on vast quantities of historical data (or training data) 617 (FIG. 6A) to build predictive models for accurate prediction on new data. Machine learning algorithm may sift through millions of records to unearth non-intuitive patterns based on data retrieved from neural networks or other sources.

In the example depicted in FIG. 7, a host platform 720 builds and deploys a machine learning model for predictive monitoring of assets 730. Here, the host platform 720 may be a cloud platform, an industrial server, a web server, a personal computer, a user device, and the like. Assets 730 can represent NDD category and treatment data and other patient-related parameters such as NDD findings at different points in time, etc.

The blockchain 621 can be used to significantly improve both a training process 702 of the machine learning model and NDD category and treatment data predictive process 704 based on a trained machine learning model. For example, in 702, rather than requiring a data scientist/engineer or other user to collect the data, historical patients' data may be stored by the assets 730 themselves (or through an intermediary, not shown) on the blockchain 621. This can significantly reduce the collection time needed by the host platform 720 when performing predictive model training. For example, using smart contracts, data can be directly and reliably transferred straight from its place of origin (e.g., practitioner's device) to the blockchain 621. By using the blockchain 621 to ensure the security and ownership of the collected data, smart contracts may directly send the data from the assets to the individuals that use the data for building a machine learning model. This allows for sharing of data among the assets 730.

The collected data may be stored in the blockchain 621 based on a consensus mechanism. The consensus mechanism pulls in (permissioned nodes) to ensure that the data being recorded is verified and accurate. The data recorded is time-stamped, cryptographically signed, and immutable. It is therefore auditable, transparent, and secure. Adding IoT devices (e.g., MRI, CT scanners, X-Ray machines, etc.) which write directly to the blockchain can increase both the frequency and accuracy of the data being recorded.

Furthermore, training of the machine learning model on the collected data may take rounds of refinement and testing by the host platform 720. Each round may be based on additional data or data that was not previously considered to help expand the knowledge of the machine learning model. In 702, the different training and testing steps (and the data associated therewith) may be stored on the blockchain 621 by the host platform 720. Each refinement of the machine learning model (e.g., changes in variables, weights, etc.) may be stored on the blockchain 621. This provides verifiable proof of how the model was trained and what data was used to train the model. Furthermore, when the host platform 720 has achieved a finally trained model, the resulting model data may be stored on the blockchain 621.

After the model has been trained, it may be deployed to a live environment where it can make optimal NDD-related predictions/decisions based on the execution of the final trained machine learning model. In this example, data fed back from the asset 730 may be input into the machine learning model and may be used to make predictions such as optimal treatment techniques based on categories. Determinations made by the execution of the machine learning model at the host platform 720 may be stored on the blockchain 621 to provide auditable/verifiable proof. As one non-limiting example, the machine learning model may predict optimal NDD-related techniques to a part of the asset 730. The data behind this decision may be stored by the host platform 720 on the blockchain 621. In one embodiment, the features and/or the actions described and/or depicted herein can occur on or with respect to the blockchain 621.

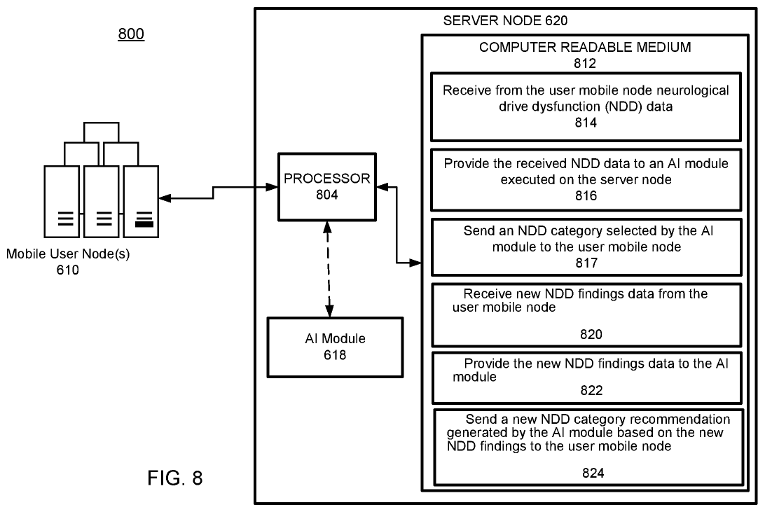

FIG. 8 illustrates network architecture of a NDD treatment system and detailed functionality of a server node that may be used by the example embodiments.

Referring to FIG. 8, the example network 800 includes the server node 620 connected to user mobile node(s) 610 over a wireless network. In one embodiment, the server node 620 may host an AI module 618. Multiple other user nodes may be connected to the server node 620. While this example describes in detail only one server node 620, multiple such nodes may be used as a cloud service. It should be understood that the server node 620 may include additional components and that some of the components described herein may be removed and/or modified without departing from a scope of the server node 620 disclosed herein. The server node 620 may be a computing device or a server computer, or the like, and may include a processor 804, which may be a semiconductor-based microprocessor, a central processing unit (CPU), an application specific integrated circuit (ASIC), a field-programmable gate array (FPGA), and/or another hardware device. Although a single processor 804 is depicted, it should be understood that the server node 620 may include multiple processors, multiple cores, or the like, without departing from the scope of the server node 620 system.

The server node 620 may also include a non-transitory computer readable medium 812 that may have stored thereon machine-readable instructions executable by the processor 804. Examples of the machine-readable instructions are shown as 814-824 and are further discussed below. Examples of the non-transitory computer readable medium 812 may include an electronic, magnetic, optical, or other physical storage device that contains or stores executable instructions. For example, the non-transitory computer readable medium 812 may be a Random Access memory (RAM), an Electrically Erasable Programmable Read-Only Memory (EEPROM), a hard disk, an optical disc, or other type of storage device.

The processor 804 may fetch, decode, and execute the machine-readable instructions 814 to receive from the user mobile node 610 neurological drive dysfunction (NDD) data. The processor 804 may fetch, decode, and execute the machine-readable instructions 816 to provide the received NDD data to an AI module 618 executed on the server node 620. The processor 804 may fetch, decode, and execute the machine-readable instructions 817 to send an NDD category selected by the AI module 618 to the user mobile node 610. The processor 804 may fetch, decode, and execute the machine-readable instructions 820 to receive new NDD findings data from the user mobile node 610. The processor 804 may fetch, decode, and execute the machine-readable instructions 822 to provide the new NDD findings data to the AI module 618. The processor 804 may fetch, decode, and execute the machine-readable instructions 824 to send a new NDD category recommendation generated by the AI module 618 based on the new NDD findings to the user mobile node 610.

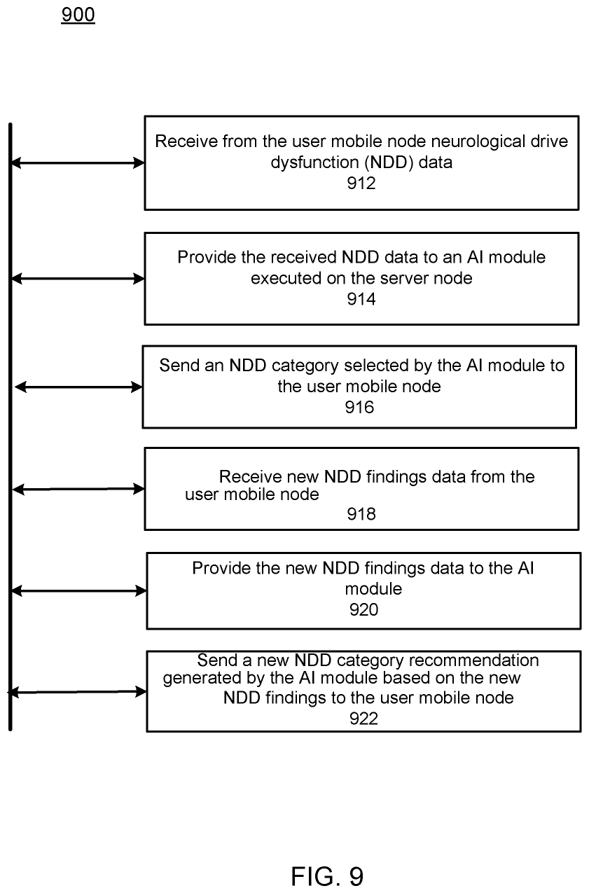

FIG. 9 illustrates a flowchart of a method executed by the server node, according to example embodiments.

It should be understood that method 900 depicted in FIG. 9 may include additional operations and that some of the operations described therein may be removed and/or modified without departing from the scope of the method 900. The description of the method 900 is also made with reference to the features depicted in FIG. 8 for purposes of illustration. Particularly, the server node 620 may execute some or all of the operations included in the method 900.

With reference to FIG. 9, at block 912, the server node 620 may receive from the user mobile node neurological drive dysfunction (NDD) data. At block 914, the server node 620 may provide the received NDD data to an AI module executed on the server node. At block 916, the server node 620 may send an NDD category selected by the AI module to the user mobile node. At block 918, the server node 620 may receive new NDD findings data from the user mobile node. At block 920, the server node 620 may provide the new NDD findings data to the AI module. At block 922, the send a new NDD category recommendation generated by the AI module based on the new NDD findings to the user mobile node.

The above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

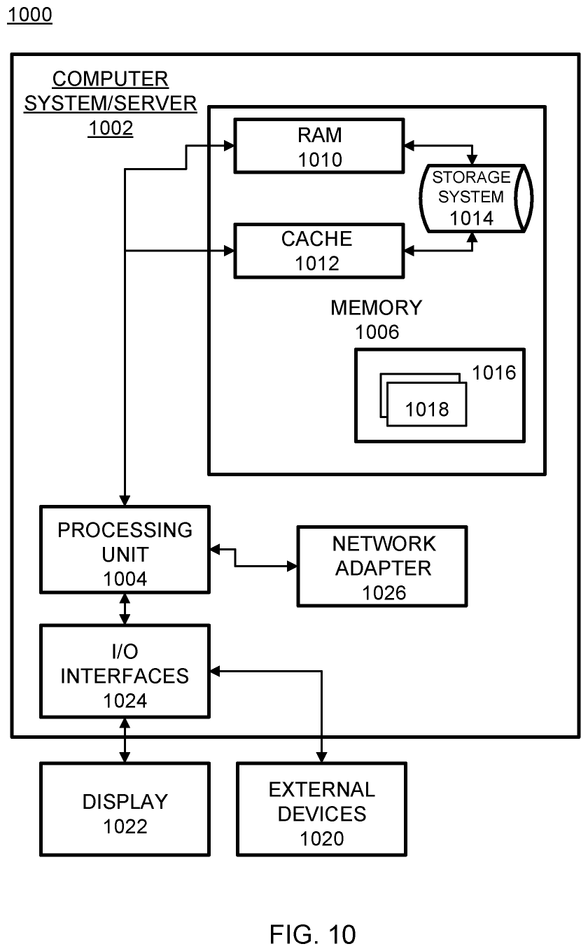

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components. For example, FIG. 10 illustrates an example computer system/server node 1000, which may represent or be integrated in any of the above-described components, etc.

The above embodiments may be implemented in hardware, in a computer program executed by a processor, in firmware, or in a combination of the above. A computer program may be embodied on a computer readable medium, such as a storage medium. For example, a computer program may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative, the processor and the storage medium may reside as discrete components.

FIG. 10 illustrates an example server node 1100 that supports one or more of the example embodiments described and/or depicted herein. The server node 1000 comprises a computer system/server 1002, which is operational with numerous other general purpose or special purpose computing system environments or configurations. Examples of well-known computing systems, environments, and/or configurations that may be suitable for use with computer system/server 1002 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system/server 1002 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 1002 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 10, computer system/server 1002 in the server node 1000 is shown in the form of a general-purpose computing device. The components of computer system/server 1002 may include, but are not limited to, one or more processors or processing units 1004, a system memory 1006, and a bus that couples various system components including system memory 1006 to processor 1004.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 1002 typically includes a variety of computer system readable media. Such media may be any available media that is accessible by computer system/server 1002, and it includes both volatile and non-volatile media, removable and non-removable media. System memory 1006, in one embodiment, implements the flow diagrams of the other figures. The system memory 1006 can include computer system readable media in the form of volatile memory, such as random-access memory (RAM) 410 and/or cache memory 1012. Computer system/server 1002 may further include other removable/non-removable, volatile/non-volatile computer system storage media. By way of example only, storage system 1014 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to the bus by one or more data media interfaces. As will be further depicted and described below, memory 1006 may include at least one program product having a set (e.g., at least one) of program modules that are configured to carry out the functions of various embodiments of the application.

Program/utility 1016, having a set (at least one) of program modules 1018, may be stored in memory 1006 by way of example, and not limitation, as well as an operating system, one or more application programs, other program modules, and program data. Each of the operating system, one or more application programs, other program modules, and program data or some combination thereof, may include an implementation of a networking environment. Program modules 1018 generally carry out the functions and/or methodologies of various embodiments of the application as described herein.

As will be appreciated by one skilled in the art, aspects of the present application may be embodied as a system, method, or computer program product. Accordingly, aspects of the present application may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present application may take the form of a computer program product embodied in one or more computer readable medium(s) having computer readable program code embodied thereon.

Computer system/server 1002 may also communicate with one or more external devices 1020 such as a keyboard, a pointing device, a display 1022, etc.; one or more devices that enable a user to interact with computer system/server 1002; and/or any devices (e.g., network card, modem, etc.) that enable computer system/server 1002 to communicate with one or more other computing devices. Such communication can occur via I/O interfaces 1024. Still yet, computer system/server 1002 can communicate with one or more networks such as a local area network (LAN), a general wide area network (WAN), and/or a public network (e.g., the Internet) via network adapter 1026. As depicted, network adapter 1026 communicates with the other components of computer system/server 1002 via a bus. It should be understood that although not shown, other hardware and/or software components could be used in conjunction with computer system/server 1002. Examples, include, but are not limited to: microcode, device drivers, redundant processing units, external disk drive arrays, RAID systems, tape drives, and data archival storage systems, etc.

Although an exemplary embodiment of at least one of a system, method, and non-transitory computer readable medium has been illustrated in the accompanied drawings and described in the foregoing detailed description, it will be understood that the application is not limited to the embodiments disclosed, but is capable of numerous rearrangements, modifications, and substitutions as set forth and defined by the following claims. For example, the capabilities of the system of the various figures can be performed by one or more of the modules or components described herein or in a distributed architecture and may include a transmitter, receiver or pair of both. For example, all or part of the functionality performed by the individual modules, may be performed by one or more of these modules. Further, the functionality described herein may be performed at various times and in relation to various events, internal or external to the modules or components. Also, the information sent between various modules can be sent between the modules via at least one of: a data network, the Internet, a voice network, an Internet Protocol network, a wireless device, a wired device and/or via plurality of protocols. Also, the messages sent or received by any of the modules may be sent or received directly and/or via one or more of the other modules.

One skilled in the art will appreciate that a “system” could be embodied as a personal computer, a server, a console, a personal digital assistant (PDA), a cell phone, a tablet computing device, a smartphone or any other suitable computing device, or combination of devices. Presenting the above-described functions as being performed by a “system” is not intended to limit the scope of the present application in any way but is intended to provide one example of many embodiments. Indeed, methods, systems and apparatuses disclosed herein may be implemented in localized and distributed forms consistent with computing technology.

It should be noted that some of the system features described in this specification have been presented as modules, in order to more particularly emphasize their implementation independence. For example, a module may be implemented as a hardware circuit comprising custom very large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together but may comprise disparate instructions stored in different locations which, when joined logically together, comprise the module and achieve the stated purpose for the module. Further, modules may be stored on a computer-readable medium, which may be, for instance, a hard disk drive, flash device, random access memory (RAM), tape, or any other such medium used to store data.

Indeed, a module of executable code could be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network.

It will be readily understood that the components of the application, as generally described and illustrated in the figures herein, may be arranged and designed in a wide variety of different configurations. Thus, the detailed description of the embodiments is not intended to limit the scope of the application as claimed but is merely representative of selected embodiments of the application.

One having ordinary skill in the art will readily understand that the above may be practiced with steps in a different order, and/or with hardware elements in configurations that are different than those which are disclosed. Therefore, although the application has been described based upon these preferred embodiments, it would be apparent to those of skill in the art that certain modifications, variations, and alternative constructions would be apparent.

While preferred embodiments of the present application have been described, it is to be understood that the embodiments described are illustrative only and the scope of the application is to be defined solely by the appended claims when considered with a full range of equivalents and modifications (e.g., protocols, hardware devices, software platforms etc.) thereto.

What is claimed is:

1. A treatment system, comprising:
a processor of a server node connected to at least one user mobile node over a network;
a memory on which are stored machine readable instructions that when executed by the processor, cause the processor to:
receive from the user mobile node neurological drive dysfunction (NDD) data;
provide the received NDD data to an AI module executed on the server node;
send an NDD category selected by the AI module to the user mobile node;
receive new NDD findings data from the user mobile node;
provide the new NDD findings data to the AI module; and
send a new NDD category recommendation generated by the AI module based on the new NDD findings to the user mobile node.

2. The system of claim 1, wherein the instructions further cause the processor to receive patient condition improvement data from the user mobile node to be stored within the AI module.

3. The system of claim 2, wherein the instructions further cause the processor to receive treatment recommendations from the AI module generated based on data retrieved from a history database containing NDD categories associated with patient condition improvement data.

4. The system of claim 1, wherein the instructions further cause the processor to receive treatment recommendations from the AI module based on data retrieved from a blockchain.

5. The system of claim 1, wherein the NDD category selected by the AI module corresponds to a linked spatial summation technique.

6. The system of claim 1, wherein the new NDD category recommendation generated by the AI module corresponds to a linked temporal summation technique.

7. The system of claim 1, wherein the instructions further cause the processor to acquire a Neurological Drive Point (NDP) recommended by the AI module, wherein the NDP corresponds to a specific Neurological Response Technique to be performed by the user.

8. The system of claim 1, wherein the instructions further cause the processor to acquire Neurological Drive Zone (NDZ) recommended by the AI module, wherein the NDZ corresponds to a specific Neurological Response Technique to be performed by the user.

9. The system of claim 1, wherein the instructions further cause the processor to acquire Neurological Drive Chain (NDC) recommended by the AI module, wherein the NDC corresponds to a specific Neurological Response Technique to be performed by the user.

10. A method, comprising:
receiving, by a server node, neurological drive dysfunction (NDD) data from a user mobile node;
providing, by the server node, the received NDD data to an AI module hosted on the server node;
sending, by the server node, an NDD category selected by the AI module to the user mobile node;
receiving, by the server node, new NDD findings data from the user mobile node;
providing, by the server node, the new NDD findings data to the AI module; and
sending a new NDD category recommendation generated by the AI module based on the new NDD findings to the user mobile node.

11. The method of claim 10, further comprising receiving patient condition improvement data from the user mobile node to be stored within the AI module.

12. The method of claim 10, further comprising receiving treatment recommendations from the AI module generated based on data retrieved from a history database containing NDD categories associated with patient condition improvement data.

13. The method of claim 10, further comprising receiving treatment recommendations from the AI module based on data retrieved from a blockchain.

14. The method of claim 10, wherein the NDD category selected by the AI module corresponds to a linked spatial summation technique.

15. The method of claim 10, wherein the new NDD category recommendation generated by the AI module corresponds to a linked temporal summation technique.

16. The method of claim 10, further comprising acquiring a Neurological Drive Point (NDP) recommended by the AI module, wherein the NDP corresponds to a specific Neurological Response Technique to be performed by the user.

17. The method of claim 10, further comprising acquiring Neurological Drive Zone (NDZ) recommended by the AI module, wherein the NDZ corresponds to a specific Neurological Response Technique to be performed by the user.

18. The method of claim 10, further comprising acquiring Neurological Drive Chain (NDC) recommended by the AI module, wherein the NDC corresponds to a specific Neurological Response Technique to be performed by the user.
